(19) United States
(12) Patent Application Publication
(10) Pub. No.: US 2009/0138093 A1
(43) Pub. Date: May 28, 2009

(54) SYSTEM AND METHOD FOR ESOPHAGEAL SPHINCTER REPAIR

(75) Inventors: STEPHEN GRAHAM BELL, Rome (IT); Wayne A. Noda, Mission Viejo, CA (US)

Correspondence Address: ROGITZ & ASSOCIATES, 750 B STREET, SUITE 3120, SAN DIEGO, CA, 92101, US

(73) Assignee: MINOS MEDICAL

(21) Appl. No.: 12/274,604

(22) Filed: Nov. 20, 2008

Related U.S. Application Data
(60) Provisional application No. 60/990,481, filed on Nov. 27, 2007.

(51) Int. Cl. A61F 2/04

(52) U.S. Cl. 623/23.64; 606/108

(57) ABSTRACT

An arcuate nitinol splint with a rounded cross-section has two ends facing each other and spaced from each other. The splint is advanced through the mouth into the esophagus by an introducer device. By means of the device the splint is implanted completely into the wall of the esophageal sphincter to strengthen the sphincter. Materials other than Nitinol may be used.

This application claims priority from U.S. provisional patent application 60/990,481, filed Nov. 27, 2007.

I. Field of the Invention

The application relates generally to repairing the esophageal or cardiac sphincter located at the gastroesophageal junction.

II. Background of the Invention

Gastro esophageal reflux disease (GERD), in which contents in the stomach pass back (“reflux”) into the esophagus, is primarily caused by a weakened esophageal sphincter. The esophageal sphincter is a circular muscle, essentially a one-way valve, at the bottom end of the esophagus that, when functioning properly in the absence of nausea, allows food to pass from the esophagus into the stomach while preventing stomach contents from passing back into the esophagus.

GERD can be treated by dietary changes, medicine, and when these treatments are insufficient, by surgery. For example, a procedure known as “fundoplication” has been introduced in which the upper curve of the stomach (the fundus) is wrapped around the esophagus and sewn into place so that the lower portion of the esophagus passes through a small tunnel of stomach muscle. This surgery strengthens the esophageal sphincter, which stops acid from backing up into the esophagus as easily. However, as understood herein the surgery is invasive even when executed laparoscopically, raising the risk of complications including infection by resistant strains of bacteria that attend all invasive procedures.

Implants have been introduced that surround the esophagus from the outside to grip it. As understood by present principles, not only does placement of such implants entail invasive surgery, but such implants can cause the swallowing disorder known as “dysphagia”. Moreover, the external implants typically can move on the esophagus, eroding tissue and in extreme cases causing death.

### SUMMARY OF THE INVENTION

A device includes an arcuate non-flaccid splint formed with two ends facing each other. The splint is configured for advancement through a patient's mouth into the esophagus by an introducer device. The splint is configured for implantation completely into the wall of the esophageal sphincter to strengthen the sphincter (intramural implantation). To this end, the splint is biased toward a narrow configuration, wherein the splint urges the wall of the sphincter inwardly. However, the splint can be moved by food passing from the esophagus into the stomach to a wide configuration wherein the ends are spaced from each other such that the sphincter can open sufficiently to allow the food to enter the stomach. This mimics and reinforces the natural tendency of the sphincter, resulting in both an anatomic and physiologic repair.

The splint may be made of Nitinol or other material, e.g., polyolefin, acetal, or teflon. If desired, the splint can be drug-eluting. Also, the splint can be hollow and can be formed with at least one opening. In example embodiments the splint has a rounded cross-section, and the splint may have a smooth exterior or an externally barbed or textured exterior for enhancing tissue engagement.

In another aspect, an introducer device includes a device body advanceable through the mouth of a patient into the esophagus. The body is configured to hold at least one esophageal sphincter splint. The device with splint is configured for forming a passage in the wall of the esophageal sphincter for placement of the splint therein. A stop member is positioned on the body a predetermined distance from the splint. The stop member is configured for abutting the esophagus-stomach fundus junction to thereby securely locate the splint at the esophageal sphincter.

In one example, the stop member is an inflatable balloon juxtaposed with a distal end of the device body. In another example, the stop member is a distal L-shaped member forming a bight with the device body. The device can be advanced into the stomach and retracted to trap a portion of the fundus and esophagus in the bight.

In another aspect, a method includes advancing a tightening element, such as a splint or an elastic suture thread, through the mouth of a patient to the esophageal sphincter. The method then includes implanting the tightening element inside the wall of the esophageal sphincter to urge the sphincter closed. The tightening element is sufficiently flexible to permit the sphincter to open when food passes through the sphincter into the stomach.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a side view of an example esophageal sphincter splint in the narrow (biased) configuration;

FIG. 2 is a side view of the splint in the wide configuration;

FIG. 3 is a cross-sectional view of the splint as seen along the line 3-3 in FIG. 1;

FIG. 4 is a cross-sectional view of an alternate hollow splint as would be seen along the line 3-3 in FIG. 1;

FIG. 5 is a cross-sectional view of the splint embedded within the esophageal sphincter;

FIG. 6 is a perspective view of a first introducer device for implanting the splint in the esophageal sphincter;

FIG. 7 is a side view in partial cross-section showing the device of FIG. 6 advanced through the mouth and esophagus into the stomach;

FIG. 8 is a side view in partial cross-section showing the device of FIG. 6 pulled back up to trap the fundus against the esophagus;

FIG. 9 is a larger side view showing the stop member of the device in FIG. 6 abutting the esophagus-stomach fundus junction to thereby securely locate the tunneler element at the esophageal sphincter;

FIG. 10 is a side view of a hollow splint;

FIG. 11 is a side view of a barbed splint;

FIG. 12 is a schematic view of an alternate introducer device for implanting the splint, with both positioning balloons inflated at the desired locations to position the splint at the esophageal sphincter;

FIG. 13 is a top plan view of the tunneler element of the device of FIG. 12 in the housed configuration, with the catheter body omitted for clarity;

FIGS. 14-16 are top plan views of the tunneler element shown in FIG. 13 in progressive stages of operation;

FIG. 17 is a perspective view of the tunneler element with push rod, showing the push rod in the vertical plane and the tunneler element in the horizontal plane.

FIG. 18 is a schematic view of an alternate deployment mechanism with alternate tightening element that is established by suture thread; and

FIG. 19 is a schematic view of the assembly shown in FIG. 18, with the suture thread cinched.

### DETAILED DESCRIPTION OF THE PREFERRED EMBODIMENT

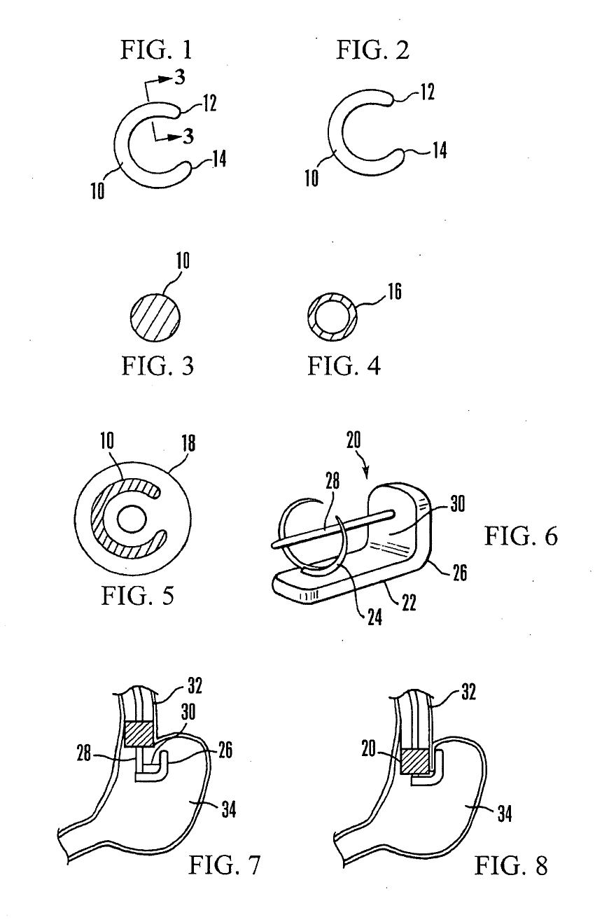

Referring initially to FIGS. 1 and 2, an arcuate non-flaccid splint 10 is shown that is formed with two ends 12, 14 facing each other. The arcuate shape of the splint 10 in its biased configuration may define a U-shape or it may define an arc of a circle greater than 180 degrees and less than 360 degrees, although it may be coiled inside the below-described introducer device in more than 360 degrees. As described in greater detail below, the splint 10 is configured for advancement through a patient's mouth into the esophagus by an introducer device for implantation completely into the wall of the esophageal sphincter to strengthen the sphincter.

As shown in cross-reference to FIGS. 1 and 2, the splint 10 is biased toward a narrow configuration (FIG. 1) in which the ends 12, 14 are spaced from each other a first distance, with the splint 10 being movable to a wide configuration (FIG. 2) in which the ends 12, 14 are spaced from each other by a greater distance than in the narrow configuration. With this structure, owing to its material bias the splint, when horizontally implanted into the wall of the esophageal sphincter, urges the wall of the sphincter inwardly. However, because the splint 10 can be moved relatively easily to the wide configuration by food passing from the esophagus into the stomach, the sphincter can open sufficiently to allow the food to enter the stomach. This sphincter opening is a result of pressure created by peristalsis of the esophagus which forces food against the sphincter.

In an example embodiment the splint 10 advantageously is made of shape memory metal such as Nitinol. Other materials may be used, e.g., polyolefin, acetal, or teflon. Furthermore, the splint 10 may be coated with a drug or otherwise be drug-eluting for delivering, e.g., anti-inflammatory drugs.

As shown in FIG. 3, the splint 10 may have around (circular) cross-section and maybe solid. Or, as shown in FIG. 4 a splint 16 that is in all substantial respects identical to the splint 10 shown in FIGS. 1-3 may be hollow.

Accordingly and turning to FIG. 5, the splint 10 may be advanced through the mouth of a patient to the esophageal sphincter 18 and then implanted entirely (“intramurally”) into the wall of the sphincter as shown. The splint 10 can be implanted horizontally inside the wall of the esophageal sphincter 18. Owing to the above-described material bias the splint 10 urges the sphincter 18 closed, but is sufficiently flexible to permit the sphincter to open when food passes through the sphincter into the stomach.

FIGS. 6-9 illustrate an example introducer device 20 for implanting the splint 10. In the example implementation shown in FIG. 6, the device 20 includes a device body 22 that is advanceable through the mouth of a patient into the esophagus. A preferably arcuate tunneler element 24 that may be established by the splint 10 itself or that maybe separate from the splint 10 and hold or otherwise be juxtaposed with the splint 10 is configured for forming a round, generally horizontal passage in the wall of the esophageal sphincter for placement of the splint. When provided as an element separate from the splint the tunneler element 24 shown in FIG. 6 may be affixed at its center point to the body 22. Also, if desired the tunneler element 24 may be coated with or otherwise elude an analgesic drug.

When the splint 10 is used as the tunneling element, the splint 10, which is biased to its preset curved shape, can be loaded in the device in a more open configuration, indeed in an almost a straight configuration. As the splint 10 is driven out by a rod pusher or other mechanism, e.g., small rollers, the splint 10 starts to curve assume its biased (non-constrained) configuration. In such an embodiment the splint can have a sharpened or pointed distal tip.

Furthermore, the thermal setting characteristics of Nitinol may be used to load the splint in one shape then cause it to assume another shape upon application of heat.

The body 22 is formed with or attached to a distal L-shaped stop member 26. The distance between the stop member 26 and the tunneling element 24 is established to approximate the distance between the esophageal sphincter and the junction of the fundus and esophagus as will be illustrated further below.

A pin 28 or other vertical support may extend proximally from the L-shaped stop member 26 as shown. Accordingly, as perhaps best shown in FIG. 7 a bight 30 is established between the pin 28 and L-shaped stop member 26.

With the above in mind, as shown in FIG. 7 the stop member 26 can be advanced entirely through the esophagus 32 into the stomach 34, with or without visualization. When visualization is desired, an endoscope may be provided on the device 20. Alternatively, ultrasound imaging or fluoroscopy or other visualization modality can be used.

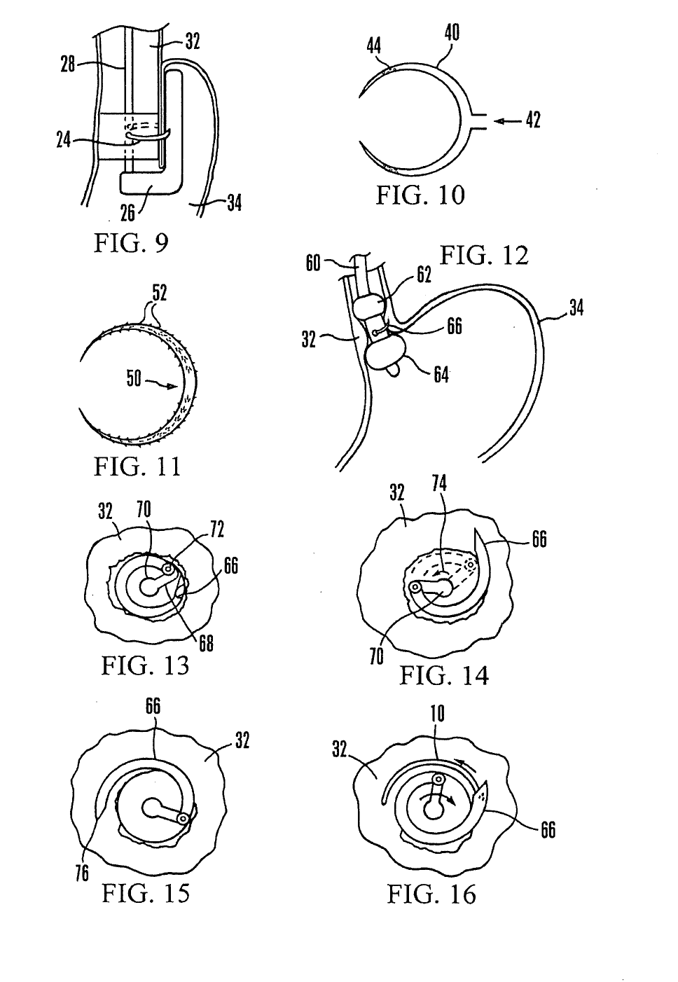

The device 20 may then be retracted proximally as shown in FIGS. 8 and 9 until the bottom of the “L” abuts the junction of the esophagus with the fundus, trapping a portion of the fundus and esophagus in the bight 30. The tunneler 24 is then rotated by, e.g., rotating the device 20 to pierce the wall of the esophagus, in effect to form a passage in the wall. One or more splints, which may be housed within the tunneler 24, can then be advanced out of the tunneler 24 by, e.g., pushing the splints out or reversing rotation of the tunneler 24 once an end of a protruding splint has gripped the tissue.

It may now be appreciated that by establishing the distance between the tunneler 24 and stop member 26, and by causing the stop member 26 to abut the esophagus-stomach fundus junction, the tunneler element 24 is securely located at the esophageal sphincter. Furthermore, by appropriately establishing the depth of the bight 30, the depth into the sphincter wall at which the splint is implanted is established.

As mentioned above, a hollow splint 40 may be provided, in which case a fluid infusion opening 42 may be formed in the splint to infuse fluid into the hollow core of the splint. One or more fluid exhaust ports 44 may also be formed in the splint. With this stricture fluid can be infused into the splint and the pressure monitored to determine proper splint placement, with a slowly decaying pressure indicating proper placement and with a quickly decaying pressure indicating improper placement (i.e., not entirely intramurally into the wall of the sphincter). Alternatively, radiological contrast media may be used as the fluid and visualized radiologically to determine leakage and, hence, proper splint placement. Methyl blue can alternatively be used to visually look for leaks.

FIG. 11 shows that if desired, a splint 50 may be formed with small external barbs 52 for gripping tissue. The barbs may be established by a textured surface of the splint 50 effected using, e.g., a diamond cut.

FIGS. 12-17 show an alternate introducer device that includes a delivery catheter 60 to which a proximal and distal balloon 62, 64 may be attached. The balloons 62, 64 are inflatable through the catheter 60. Between the balloons 62, 64 a hollow tunneler element 66 can be disposed and can protrude outside the catheter 60 as shown.

With this structure, the balloons 62, 64 can be deflated and then the device advanced through the mouth and esophagus 32 until the distal balloon 64 clears the esophagus and is disposed in the stomach 34. The distal balloon 64 can then be inflated and the device retracted proximally until the distal balloon 64 abuts the junction of the esophagus and fundus as shown in FIG. 12. When a proximal balloon 62 is provided, it may then be inflated to anchor the device at the appropriate location in the esophagus.

FIGS. 13-16 illustrate how the splint 10 shown in FIGS. 1-3 may be deployed into the wall of esophageal sphincter using the device shown in FIGS. 12-17. The tunneler element 66 can be a resilient flexible tubular device that is trapped in the catheter 60 in a coil-like configuration. In the example non-limiting embodiment shown, the tunneler 66 is joined to a horizontal arm 68 of a vertical rotatable hollow shaft 70 within the catheter at a pivot point 72. As the shaft 70 is rotated as indicated by the arrow 74 in FIG. 14, the tunneler 66, which protrudes from the catheter 60 as described above, is pushed from the configuration shown in phantom in FIG. 14 to the configuration shown in solid. As the tunneler 66 emerges from the catheter, owing to its material bias it uncoils (FIG. 15), piercing the wall of the sphincter. To this end, the tunneler 66 may be formed with a sharpened distal tip 76.

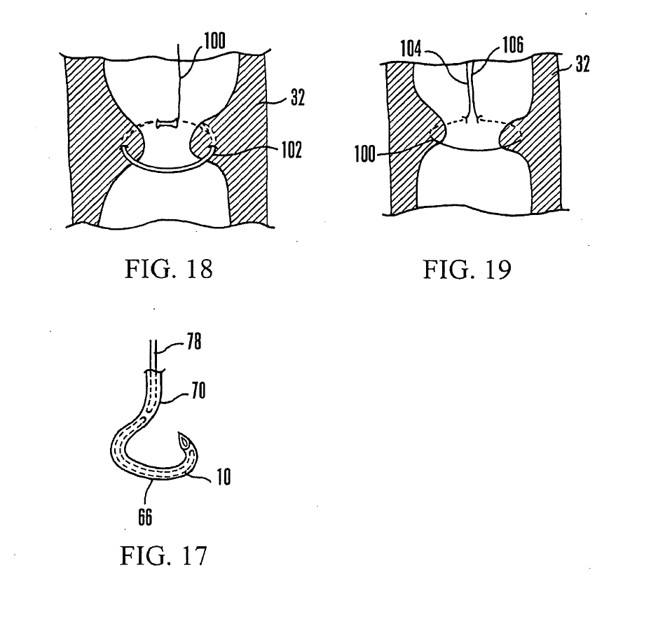

Then, in cross-reference to FIGS. 16 and 17 a flexible push rod 78 that may be made of nitinol can be advanced through the shalt 70 against one or more splints 10 (shown in phantom in FIG. 17) that are housed within the tunneler 66. With the push rod 78 against the splint 10, the tunneler 66 may be rotated in the reverse direction as shown in FIG. 16, leaving the splint 10 in place inside the wall of the sphincter. Once the splint completely clears the tunneler 66, the introducer device may be retracted from the patient.

FIGS. 18 and 19 show that instead of a non-flaccid splint, a tightening element according to present principles may be established by an elastic suture thread 100. To deploy the thread 100 within the wall of the esophageal sphincter 32, the thread 100 can be loaded into a hollow tunneler 102, with an end of the thread extending back through the introducer device and out of the patient's mouth. The tunneler 102 may function in accordance with above principles to pierce the wall of the sphincter, leading the suture thread through the wall until the suture thread 100 generally forms a circle inside the wall. As shown in FIG. 19, the device is then retracted back through the patient's mouth such that both ends 104, 106 of the thread 100 extend outside the patient's mouth. The ends 104, 106 can be tensioned and knotted by advancing an extracorporeal knot down into the sphincter in accordance with suture knotting principles known in the art. Owing to its elasticity, the thread 100 can expand within the wall to allow food to pass from the esophagus into the stomach.

While the particular SYSTEM AND METHOD FOR ESOPHAGEAL SPHINCTER REPAIR is herein shown and described in detail, it is to be understood that the subject matter which is encompassed by the present invention is limited only by the claims. For example, while natural orifice placement of the splint is envisioned, the splint alternatively may be placed laparoscopically.

What is claimed is:

1. Device, comprising:
an arcuate non-flaccid splint formed with two ends facing each other, the splint configured for advancement through a patient's mouth into the esophagus by an introducer device, the splint configured for implantation completely into the wall of the esophageal sphincter to strengthen the sphincter, the splint being biased toward a narrow configuration, wherein the splint urges the wall of the sphincter inwardly, the splint being movable by food passing from the esophagus into the stomach to a wide configuration wherein the ends are spaced from each other such that the sphincter can open sufficiently to allow the food to enter the stomach.

2. The device of claim 1, wherein the splint is made of Nitinol.

3. The device of claim 1, wherein the splint is made of polyolefin, acetal, or teflon.

4. The device of claim 1, wherein the splint is drug-eluting.

5. The device of claim 1, wherein the splint is hollow and is formed with at least one opening.

6. The device of claim 1, wherein the splint has a rounded cross-section.

7. The device of claim 1, wherein the splint includes external barbs for enhancing tissue engagement.

8. An introducer device comprising:
a device body advanceable through the mouth of a patient into the esophagus, the body configured to hold at least one esophageal sphincter splint;
the device with splint configured for forming a passage in the wall of the esophageal sphincter for placement of the splint therein; and
at least one stop member positioned on the body a predetermined distance from the splint and configured for abutting the esophagus-stomach fundus junction to thereby securely locate the splint at the esophageal sphincter.

9. The device of claim 8, wherein the stop member is an inflatable balloon juxtaposed with a distal end of the device body.

10. The device of claim 8, wherein the stop member is a distal L-shaped member forming a bight with the device body, the device being advanceable into the stomach and retractable to trap a portion of the fundus and esophagus in the bight, a depth of the bight being established to establish a depth into the sphincter wall at which the splint is implanted.

11. The device of claim 8, comprising a tunneler element separate from the splint and holding the splint, wherein the tunneler element is rotatable to pierce the wall of the esophagus.

12. Method comprising:
advancing a tightening element through the mouth of a patient to the esophageal sphincter; and
implanting the tightening element inside the wall of the esophageal sphincter to urge the sphincter closed, the tightening element being sufficiently flexible to permit the sphincter to open when food passes through the sphincter into the stomach.

13. The method of claim 12, wherein the tightening element is at least one non-flaccid splint.

14. The method of claim 12, wherein the tightening element is established by a suture thread.

15. The method of claim 13, wherein the splint is an arcuate non-flaccid splint formed with two ends facing each other, the splint being biased to a narrow configuration, wherein the splint urges the wall of the sphincter inwardly, the splint being movable by food passing from the esophagus into the stomach to a wide configuration wherein the ends are spaced from each other such that the sphincter can open sufficiently to allow the food to enter the stomach.

16. The method of claim 15, wherein the splint is made of Nitinol.

17. The method of claim 15, wherein the splint is made of polyolefin, acetal, or teflon.

18. The method of claim 15, wherein the splint is drug-eluting.

19. The method of claim 15, wherein the splint is hollow and is formed with at least one opening.

20. The method of claim 15, wherein the splint has a rounded cross-section.

21. The method of claim 15, wherein the splint includes external barbs for enhancing tissue engagement.
